(19) United States
(12) Patent Application Publication
(10) Pub. No.: US 2009/0068295 A1
(43) Pub. Date: Mar. 12, 2009

(54) Composition for treating cancer and method of using the same

(75) Inventor: Jeng-Shu Wei, Tao-Yuan (TW)

Correspondence Address: BACON & THOMAS, PLLC, 625 SLATERS LANE, FOURTH FLOOR, ALEXANDRIA, VA, 22314-1176, US

(73) Assignee: Chang Gung University, Tao-Yuan (TW)

(21) Appl. No.: 12/000,354

(22) Filed: Dec. 12, 2007

(30) Foreign Application Priority Data
Sep. 10, 2007 (TW) 096133656

(51) Int. Cl. A61K 36/82; A61K 31/495; A61P 35/00

(52) U.S. Cl. 424/729; 514/256

(57) ABSTRACT

The present invention provides a composition, which includes an effective amount of DNA damaging agent and an effective amount of tea polyphenols. Particularly, the preferred DNA damaging agent is 5-Fluorouracil (5-FU), and the preferred tea polyphenols is Epigallocatechin gallate (EGCG).

### BACKGROUND OF THE INVENTION

1. Field of the invention

The present invention relates generally to a composition, and more particularly, to a composition for treating cancer and method of using the same.

2. Description of the prior art

With the aggravation of environment, people frequently contacts with carcinogenic substances, such as smoke, alcoholic drink, radiation, air, water, merchandise, foods with toxins and virus in every daily life. Therefore, in many countries, cancer or malignant tumor has become one of the major causes of death. Based on statistic data, the death rate caused by cancer or malignant tumor per 100,000 population of the world is about 100 to 350 persons.

Briefly, cancers or malignant tumors are major caused by abnormal control of cell growth and proliferation. In addition, the abnormal control of cell growth, cancer cells may locally invade peripheral normal tissues, also known as invasion, and may even transfer to other parts of body through the circulatory system or lymphatic system, also known as metastasis. Furthermore, cancer always could be named in accordance with the sites occurring, such as prostate cancer, lung cancer, breast cancer, colon cancer, skin cancer, gastric cancer, leukemia, or oral cancer.

Furthermore, the treatment of cancer can include surgery, chemical treatment, radiation treatment, immuno-treatment, monoclonal antibody treatment, or the other suitable treatments. The choice of the type of treatment is based on the sites of tumors, the malignant level, progress level, and the condition of patients. Wherein, the chemical treatment refers to use drugs which can kill cancer cells to treat patients. Because the major different between cancer cells and normal cells is that the cancer cells can grow and divide quickly, anti-cancer drugs often inhibits the growth of cancer cells by interrupting cell division, such as inhibiting DNA duplication or prohibiting chromosome separation.

Referring to FIG. 1 which shows the chemical structural formula of 5-Fluorouracil of the prior art. 5-FU belongs to antimetabolites which have been used for about 40 years. It is one of the most often used chemical drugs by oncology doctors, and widely used to treat cancers such as gastric cancer, colon cancer, pancreatic cancer, or breast cancer. The derivatives of 5-FU can combine with thymidylate synthase (TS) and therefore inhibit the function of TS, so that interrupt the production of thymidylate and the synthesis of DNA, and stop the division of cancer cells.

Further referring to FIG. 2 which illustrates the mechanism of 5-FU in the cell. As shown in FIG. 2, 5-FU can be transferred to 5-FdUMP and FUTP. 5-FdUMP can combine with TS, so that the accumulation of deoxyuridine triphosphates, the precursor of DNA, is unbalance, and the double strand of DNA is easily to be broken. Additionally, 5-FdUMP can be phospholated to form 5-FdUTP which can also combine to DNA and cause the break of DNA double strand.

Recently, 5-FU often been combined with other chemicals such as Leucovorin, an active metabolite of folic acid, to treat patients with cancers. Leucovorin can combine with 5-FU and TS to form a more stable complex, and then can improve the effect of 5-FU.

Although 5-FU has significant effect on treating some specific cancers, the therapeutic effect is believed to be further improved. Moreover, side effects, such as anorexia, nausea, vomit, gastritis, mucous membrane inflammation, diarrhea, or hair loss, may exist in the patients who receive the injection of 5-FU, so as to cause heavy burden on the patients.

### SUMMARY OF THE INVENTION

Accordingly, an aspect of the present invention is to provide a composition for treating cancer. Particularly, the composition of the invention can improve the curative effect of 5-FU, and further reduce the side effect caused by 5-FU.

According to a preferred embodiment, the composition of the invention includes an effective amount of a DNA damaging agent, and an effective amount of a tea polyphenols. Particularly, the DNA damaging agent preferably is 5-FU, and the tea polyphenols preferably is EGCG.

Another aspect of the present invention is to provide a method for inhibiting the growth of a tumor. The method of the invention can inhibit the growth of tumor cells, or promote apoptosis of tumor cells.

According to a preferred embodiment, the method of the invention includes the following step: contact an effective amount of a DNA damaging agent and an effective amount of a tea polyphenols with a cell of the tumor. Particularly, the DNA damaging agent preferably is 5-FU, and the tea polyphenols preferably is EGCG. Furthermore, the method of the invention can preferably inhibit the growth of mammalian tumor cells.

The aspect of the present invention will no doubt become obvious to those of ordinary skill in the art after reading the following detailed description of the preferred embodiment, which is illustrated in the various figures and drawings.

### BRIEF DESCRIPTION OF THE APPENDED DRAWINGS

FIG. 1 shows the chemical structural formula of 5-Fluorouracil of the prior art;

FIG. 2 illustrates the mechanism of 5-FU in the cell;

FIG. 3A shows the results of treating human liver cancer cell line Sk-Hep-1 with 10 μM of 5-FU, 50 μM of EGCG, 100 μM of EGCG, 250 μM of EGCG, and 500 μM of EGCG respectively;

FIG. 3B shows the results of treating human liver cancer cell line Sk-Hep-1 with 10 μM of 5-FU and 50 μM of EGCG, 100 μM of EGCG, 250 μM of EGCG, and 500 μM of EGCG respectively;

FIG. 4A shows the effect of the composition, which includes 5-FU and EGCG, of the invention on the cell cycle of human liver cancer cell line Sk-Hep-1;

FIG. 4B shows the effect of the composition, which includes 5-FU and EGCG, of the invention on the cell cycle of human liver cancer cell line Hep-G2;

FIG. 5A shows the effect of the composition, which includes 5-FU and EGCG, of the invention on the P53 protein expression of human liver cancer cell line Sk-Hep-1;

FIG. 5B shows the effect of the composition, which includes 5-FU and EGCG, of the invention on the P53 protein expression of human liver cancer cell line Hep-G2;

FIG. 6A shows the effect of the composition, which includes 5-FU and EGCG, of the invention on the viability of human leukemia cell line Jurkat T; and

FIG. 6B shows the effect of the composition, which includes 5-FU and EGCG, of the invention on the viability of human leukemia cell line K562.

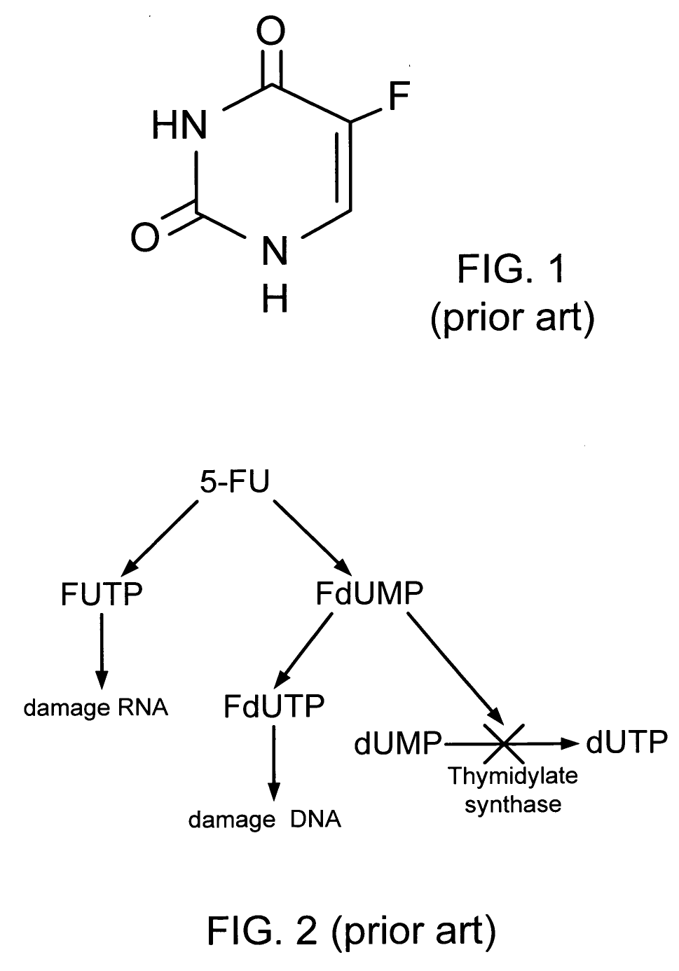

### DETAILED DESCRIPTION OF THE INVENTION

The present invention provides a composition, which includes an effective amount of a DNA damaging agent, and an effective amount of tea polyphenols. Particularly, the composition of the invention can be a pharmaceutical composition, which includes an effective amount of DNA damaging agent, and an effective amount of tea polyphenols.

The term “effective amount” as used in this application means the amount of the DNA damaging agent and the tea polyphenols that is effective to induce apoptosis in enough cells. Furthermore, the term “therapeutically effective amount” refers to an amount of the DNA damaging agent and the tea polyphenols that is effective to kill enough cells in an animal when administered into the animal. Especially, the term “therapeutically effective amount” refers to an amount of the DNA damaging agent and the tea polyphenols that is effective to kill enough tumor cells, such as liver cancer cells, lung cancer cells, leukemia cells, breast cancer cells, or colon cancer cells, in an animal or human.

Furthermore, the DNA damaging agent included in the composition of the invention preferably is 5-Fluorouracil (5-FU). The tea polyphenols included in the composition of the invention can be, but not limited to, such as Epigallocatechin gallate (EGCG), Epigallocatechin (EGC), Epicatechin gallate (ECG), or Epicatechin (EC). Preferably, the tea polyphenols included in the composition of the invention is EGCG.

Practically, the ratio of the above-mentioned DNA damaging agent to the tea polyphenols is in between 2% and 10%, such as 2%, 3%, 4%, 5%, 6%, 7%, 8%, 9%, 10%. That is to say, when the composition of the invention includes 10 μM of DNA, it also includes 100 μM to 500 μM of tea polyphenols. Preferably, the ratio of the DNA damaging agent to the tea polyphenols is about 4%. That is to say, when the composition includes 10 μM of DNA, it also includes about 250 μM of tea polyphenols.

In practice, the DNA damaging agent can be chosen as other suitable agent, and the tea polyphenols can be replaced with other suitable ingredients, but not limited to the above-mentioned examples.

In an embodiment, the composition of the invention can further includes a carrier, which can be a mixture of water and Dimethyl sulfoxide (DMSO), or a mixture of water and Dimethylformamide (DMF). Moreover, in the embodiment, the ratio of water to DMSO or DMF can be optionally adjusted to any suitable ratio.

In an embodiment, the composition of the invention can further includes suitable adjuvant, for improving the function of the DNA damaging agent. For example, the adjuvant can be Leucovorin, as mentioned above, Leucovorin can combine with 5-FU and thymidylate synthase to form a more stable complex, so as to improve the function of 5-FU.

In a preferred embodiment, the invention discloses a method for inhibiting the growth of a tumor, and the method includes the following step: contacting an effective amount of a DNA damaging agent and an effective amount of a tea polyphenols with a cell of the tumor. Particularly, the DNA damaging agent preferably is 5-FU, and the tea polyphenols can be EGCG, EGC, ECG, and EC, and preferably is EGCG.

Furthermore, as mentioned before, in an embodiment, the ratio of the above-mentioned DNA damaging agent to the tea polyphenols is in between 2% and 10%, such as 2%, 3%, 4%, 5%, 6%, 7%, 8%, 9%, 10%. Preferably, the ratio of the DNA damaging agent to the tea polyphenols is about 4%. In practice, the method of the invention can apply the effective amount of the DNA damaging agent to contact with the cell first, and then apply the effective amount of the tea polyphenols to contact with the cell. Or, the method can apply the effective amount of the tea polyphenols to contact with the cell first, and then apply the effective amount of the DNA damaging agent to contact with the cell. Certainly, the method can apply both of the effective amount of the DNA damaging agent and the effective amount of the tea polyphenols to contact with the cell at the same time.

The growth of tumor cell, also know as “transformation”, refers to the growth and proliferation of cells which loss the control of cell differentiation. Expectably, different types of transformation cells, such as sarcoma cells, melanoma cells, lymphoma cells, or other solid tumor cells, can be the potential objects of the composition of the invention. Moreover, liver cancer cell and leukemia cell are the preferred objects of the composition of the invention.

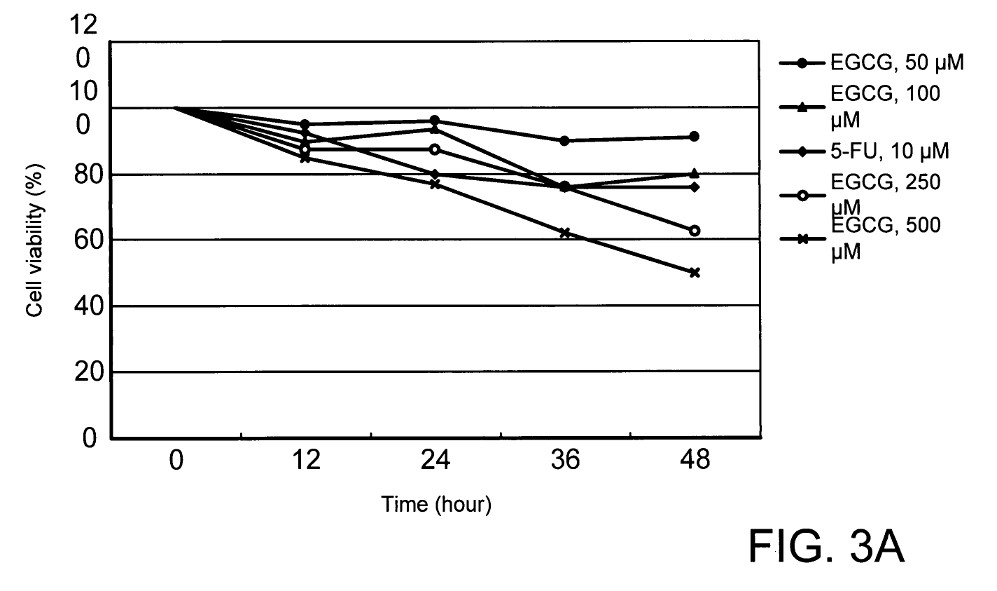

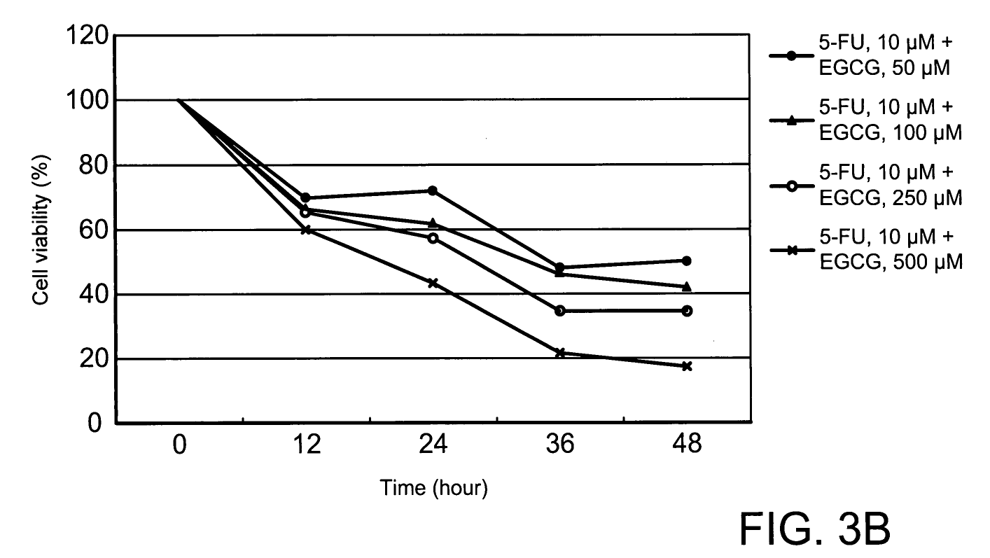

In an embodiment, the malignant cell is located in a tumor site, such as a tumor bed, in an animal. Moreover, the animal can be a mammalian, such as human, mouse, rat, or other mammalians. Therefore, in the embodiment, the method of the invention includes the step of administrating a treatment effective amount of the DNA damaging agent and the tea polyphenols in the animal. In practice, the composition of the invention can be delivered into the animal via injection, such as intravenous injection, hypodermic injection, intratumor injection and intraperitoneal injection. Referring to FIG. 3A and FIG. 3B. FIG. 3A shows the results of treating human liver cancer cell line Sk-Hep-1 with 10 μM of 5-FU, 50 μM of EGCG, 100 μM of EGCG, 250 μM of EGCG, and 500 μM of EGCG respectively. FIG. 3B shows the results of treating human liver cancer cell line Sk-Hep-1 with 10 μM of 5-FU and 50 μM of EGCG, 100 μM of EGCG, 250 μM of EGCG, and 500 μM of EGCG respectively.

As shown in FIG. 3A, the viability of Sk-Hep-1 by treating with 10 μM of 5-FU after 48 hours is about 75%, and the viability of Sk-Hep-1 by treating with 50 μM of EGCG, 100 μM of EGCG, 250 μM of EGCG, and 500 μM of EGCG after 48 hours are about 90%, 80%, 60%, and 50% respectively. Furthermore, as shown in FIG. 3B, when 10 μM of 5-FU is combined with 50 μM of EGCG, 100 μM of EGCG, 250 μM of EGCG, or 500 μM of EGCG to treat Sk-Hep-1, the viability of Sk-Hep-1 after 48 hours is obviously reduced to 50%, 40%, 35%, and 19%. That is to say, the treatment which includes both 5-FU and EGCG can largely increase the capability of 5-FU to inhibit the growth of cancer cell Sk-Hep-1.

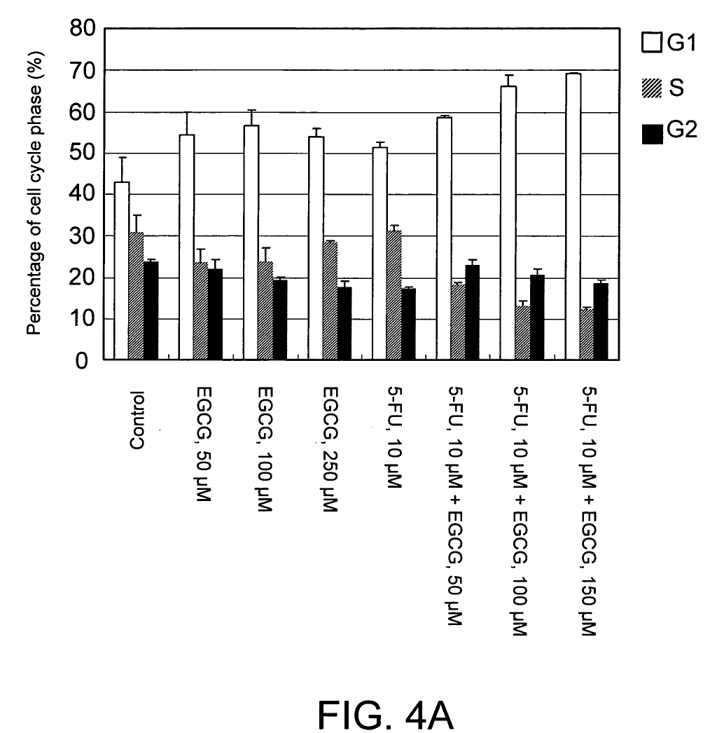

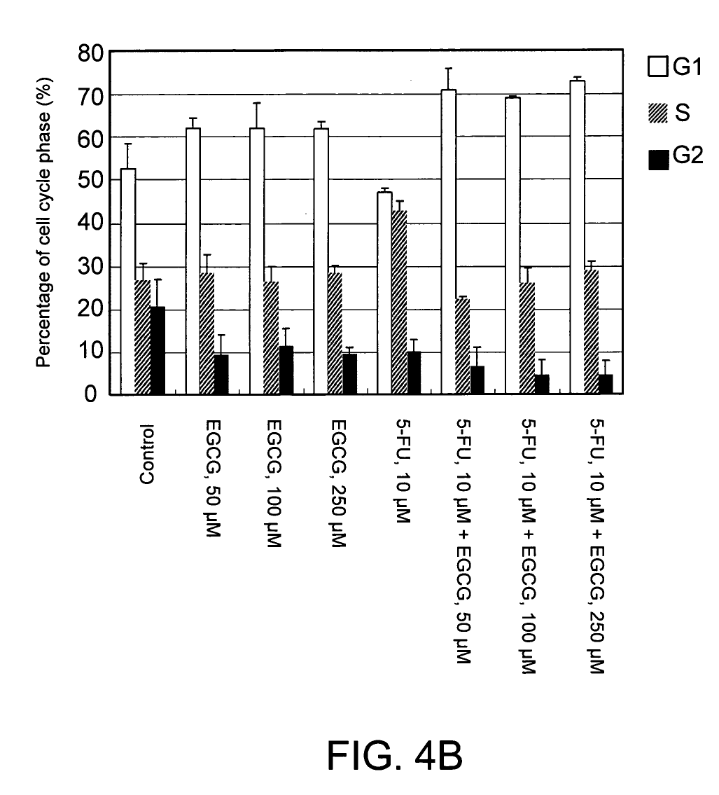

Please refer to FIG. 4A and FIG. 4B. FIG. 4A shows the effect of the composition, which includes 5-FU and EGCG of the invention on the cell cycle of human liver cancer cell line Sk-Hep-1. FIG. 4B shows the effect of the composition, which includes 5-FU and EGCG of the invention on the cell cycle of human liver cancer cell line Hep-G2. Please further refer to the following Table 1 and Table 2. Table 1 lists the detail data of FIG. 4A, and Table 2 lists the detail data of FIG. 4B.

TABLE 1

| Cell cycle phase | G1 (%) | S (%) | G2 (%) |
|---|---|---|---|
| Control | 43.04 ± 5.9 | 30.9 ± 4.2 | 23.75 ± 0.53 |
| EGCG, 50 μM | 54.41 ± 5.4 | 23.51 ± 3.2 | 22.09 ± 2.2 |
| EGCG, 100 μM | 56.60 ± 3.8 | 23.90 ± 3.1 | 19.5 ± 0.7 |
| EGCG, 250 μM | 53.92 ± 1.9 | 28.53 ± 2.3 | 17.55 ± 1.7 |
| 5-FU, 10 μM | 51.51 ± 1.1 | 31.19 ± 1.4 | 17.30 ± 0.3 |
| 5-FU, | 58.57 ± 0.6 | 18.3 ± 0.6 | 23.13 ± 1.1 |
| 10 μM + EGCG, 50 μM | | | |
| 5-FU, | 66.15 ± 2.6 | 13.21 ± 1.2 | 20.64 ± 1.4 |
| 10 μM + EGCG, | | | |
| 100 μM | | | |
| 5-FU, | 69.05 ± 0.3 | 12.43 ± 0.42 | 18.53 ± 0.73 |
| 10 μM + EGCG, | | | |
| 150 μM | | | |

TABLE 2

| Cell cycle phase | G1 (%) | S (%) | G2 (%) |
|---|---|---|---|
| Control | 52.7 ± 5.8 | 26.54 ± 4.2 | 20.76 ± 6.1 |
| EGCG, 50 μM | 62.15 ± 2.4 | 28.52 ± 4.3 | 9.33 ± 4.7 |
| EGCG, 100 μM | 62.23 ± 5.7 | 26.26 ± 3.5 | 11.51 ± 4.1 |
| EGCG, 250 μM | 61.83 ± 1.6 | 28.53 ± 1.3 | 9.63 ± 1.7 |
| 5-FU, 10 μM | 47.04 ± 1.1 | 42.68 ± 2.4 | 10.29 ± 2.5 |
| 5-FU, 10 μM + EGCG, | 70.99 ± 4.8 | 22.2 ± 0.6 | 6.8 ± 4.5 |
| 50 μM | | | |
| 5-FU, 10 μM + EGCG, | 69.14 ± 0.2 | 26.14 ± 3.4 | 4.72 ± 3.5 |
| 100 μM | | | |
| 5-FU, 10 μM + EGCG, | 72.94 ± 0.8 | 29.14 ± 1.9 | 4.59 ± 3.3 |
| 150 μM | | | |

As shown in FIG. 4A and Table 1, in the cell cycle of Sk-Hep-1, the percentage of G1, S, and G2 phases are about 43.04%, 30.9%, and 23.75% respectively. When treat the Sk-Hep-1 cells with 10 μM of 5-FU alone, the percentage of G1, S, and G2 phases are about 51.51%, 31.19%, and 17.3% respectively. From the comparison with the control, 5-FU can increase the percentage of G1 phase and reduce the percentage of G2 phase. Furthermore, when using 10 μM of 5-FU together with the EGCG to treat Sk-Hep-1 cells, the percentage of G1 phase obviously increased up to about 58.57% to 69.05%, and the percentage of S phase obviously decreased down to about 12.43% to 18.3%. From the results, 5-FU combined with EGCG can effectively interrupt the cell cycle of Sk-Hep-1 cells at G1 phase, and prevent the cell cycle from entering S phase.

Furthermore, as shown in FIG. 4B and Table 2, in the cell cycle of Hep-G2, the percentage of G1, S, and G2 phases are about 52.7%, 26.54%, and 20.76% respectively. When treat the Hep-G2 cells with 10 μM of 5-FU alone, the percentage of G1, S, and G2 phases are about 47.04%, 42.68%, and 10.29% respectively. From the comparison with the control, 5-FU can increase the percentage of S phase and reduce the percentage of G1 phase and G2 phase. Additionally, when using 10 μM of 5-FU together with the EGCG to treat Hep-G2 cells, the percentage of G1 phase obviously increased up to about 70.99% to 72.94%, and the percentage of G2 phase obviously decreased down to about 4.59% to 6.8%. From the results, 5-FU combined with EGCG can effectively interrupt the cell cycle of Hep-G2 cells at G1 phase, and prevent the cell cycle from entering S phase and G2 phase.

In summary, the treatment of 5-FU together with EGCG can largely increase the capability of 5-FU to interrupt the cell cycle of Sk-Hep-1 and Hep-G2.

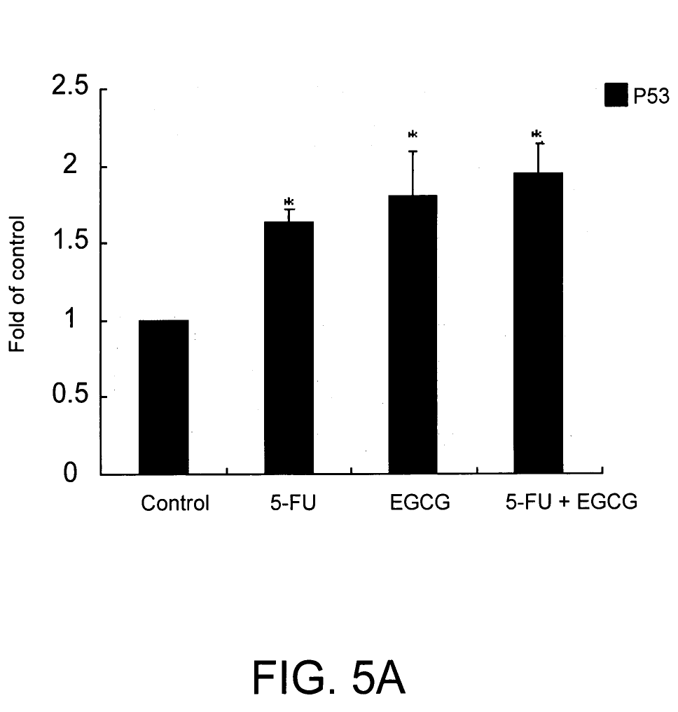

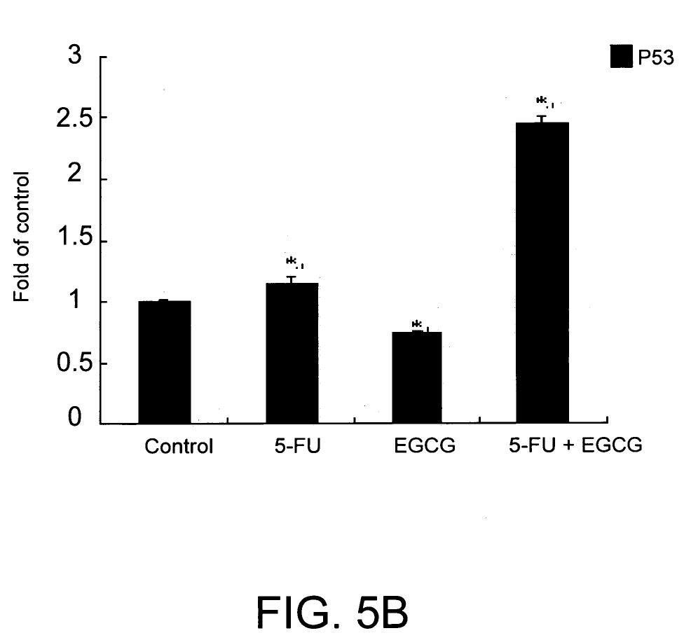

Referring to FIG. 5A and FIG. 5B. FIG. 5A shows the effect of the composition, which includes 5-FU and EGCG of the invention on the P53 protein expression of human liver cancer cell line Sk-Hep-1. FIG. 5B shows the effect of the composition, which includes 5-FU and EGCG of the invention on the P53 protein expression of human liver cancer cell line Hep-G2.

As shown in FIG. 5A, compared with control, treat Sk-Hep-1 cells with 5-FU or EGCG can increase the expression of P53 protein to 1.64-fold or 1.8-fold. Moreover, treat Sk-Hep-1 cells with the combination of 5-FU and EGCG can largely increase the expression of P53 protein to 1.95-fold.

As shown in FIG. 5B, compared with control, treat Hep-G2 cells with 5-FU can increase the expression of P53 protein to 1.64-fold. However, treat Hep-G2 with EGCG can reduce the expression of P53 protein to 0.74-fold. Moreover, treat Hep-G2 cells with the combination of 5-FU and EGCG can largely increase the expression of P53 protein to 2.46-fold.

In summary, the treatment of 5-FU together with EGCG can effectively increase the expression of P53 protein, a well-known tumor-supressor protein, so as to inhibit the growth of tumor cells.

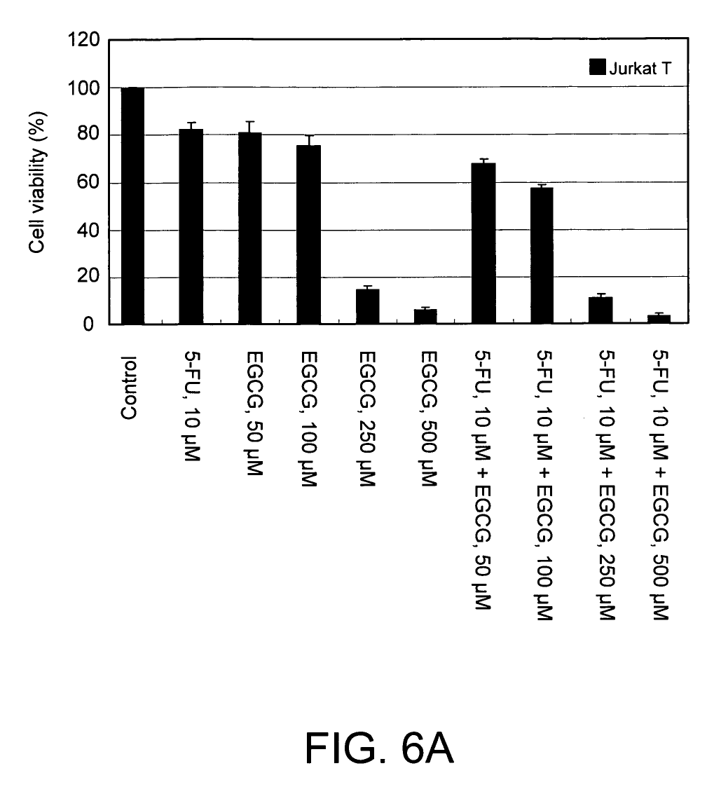

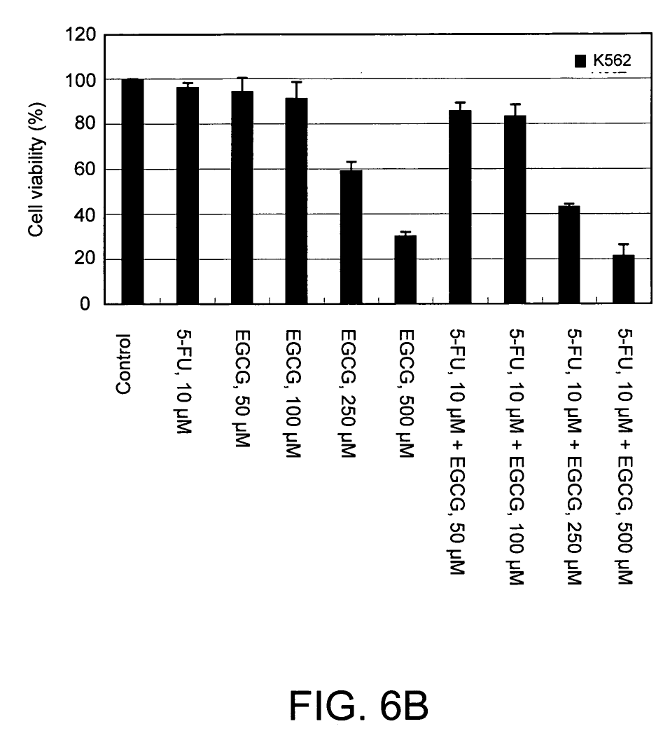

Please refer to FIG. 6A and FIG. 6B. FIG. 6A shows the effect of the composition, which includes 5-FU and EGCG of the invention on the viability of human leukemia cell line Jurkat T. FIG. 6B shows the effect of the composition, which includes 5-FU and EGCG, of the invention on the viability of human leukemia cell line K562.

As shown in FIG. 6A, the treatment of 5-FU alone can reduce the viability of Jurkat T to about 80%. Moreover, the treatment of 5-FU together with EGCG can significantly reduce the viability of Jurkat T to less than 20% (10 μM of 5-FU+250 μM of EGCG), even less than 5% (10 μM of 5-FU+500 μM of EGCG). As shown in FIG. 6B, the treatment of 5-FU alone can have almost no effect on the viability of K562. Moreover, the treatment of 5-FU together with EGCG can significantly reduce the viability of K562 to less than 40% (10 μM of 5-FU+250 μM of EGCG), even less than 20% (10 μM of 5-FU+500 μM of EGCG).

In summary, the composition of the invention can effectively reduce the viability of tumor cells, especially human liver cancer cells and leukemia cells. Furthermore, the composition of the invention can interrupt the cell cycle of cancer cells, and further induce the expression of P53 protein. Therefore, the composition of the invention can effectively inhibit the growth of tumors.

It is understood that various other modifications will be apparent to and can be readily made by those skilled in the art without departing from the scope and spirit of this invention. Accordingly, it is not intended that the scope of the claims appended hereto be limited to the description as set forth herein, but rather that the claims be construed as encompassing all the features of patentable novelty that reside in the present invention, including all features that would be treated as equivalents thereof by those skilled in the art to which this invention pertains.

1. A composition, comprising:
an effective amount of a DNA damaging agent; and
an effective amount of a tea polyphenols.

2. The composition according to claim 1, wherein the DNA damaging agent is 5-Fluorouracil (5-FU).

3. The composition according to claim 1, wherein the tea polyphenols are selected from a group consisting of Epigallocatechin gallate (EGCG), Epigallocatechin (EGC), Epicatechin gallate (ECG), and Epicatechin (EC).

4. The composition according to claim 3, wherein the tea polyphenols is EGCG.

5. The composition according to claim 1, further comprising a carrier which comprises Dimethyl sulfoxide (DMSO) or Dimethylformamide (DMF).

6. The composition according to claim 1, further comprising Leucovorin.

7. The composition according to claim 1, wherein the ratio of the DNA damaging agent to the tea polyphenols is in between 2% and 10%.

8. The composition according to claim 7, wherein the ratio of the DNA damaging agent to the tea polyphenols is about 4%.

9-20. (canceled)

21. A composition for treating liver cancer, comprising:
an effective amount of a DNA damaging agent; and
an effective amount of a tea polyphenols.

22. The composition according to claim 21, wherein the DNA damaging agent is 5-Fluorouracil (5-FU).

23. The composition according to claim 21, wherein the tea polyphenols are selected from a group consisting of Epigallocatechin gallate (EGCG), Epigallocatechin (EGC), Epicatechin gallate (ECG), and Epicatechin (EC).

24. The composition according to claim 23, wherein the tea polyphenols is EGCG.

25. The composition according to claim 21, further comprising a carrier which comprises Dimethyl sulfoxide (DMSO) or Dimethylformamide (DMF).

26. The composition according to claim 21, further comprising Leucovorin.

27. The composition according to claim 21, wherein the ratio of the DNA damaging agent to the tea polyphenols is in between 2% and 10%.

28. The composition according to claim 27, wherein the ratio of the DNA damaging agent to the tea polyphenols is about 4%.

29. The composition of claim 1 for treating leukemia.

30. The composition according to claim 29, wherein the DNA damaging agent is 5-Fluorouracil (5-FU), the tea polyphenols is selected from the group consisting of Epigallocatechin gallate (EGCG), Epigallocatechin (EGC), Epicatechin gallate (ECG), and Epicatechin (EC); and further comprising a carrier which is Dimethyl sulfoxide (DMSO) or Dimethylformamide (DMF); and wherein the ratio of the DNA damaging agent to the tea polyphenols is in between 2% and 10%.

31. The composition according to claim 30, further comprising Leucovorin.

32. The composition according to claim 30, wherein the ratio of the DNA damaging agent to the tea polyphenols is about 4%.
